(19) United States
(12) Patent Application Publication
(10) Pub. No.: US 2021/0163866 A1
(43) Pub. Date: Jun. 3, 2021

(54) CELL MEASUREMENTS AFTER ISOLATION FROM SOLUTIONS IN A MICROFLUIDIC CHANNEL

(71) Applicant: Hewlett-Packard Development Company, L.P., Spring, TX (US)

(72) Inventors: Steven Barcelo, Palo Alto, CA (US); Anita Rogacs, San Diego, CA (US); Fausto D'Apuzzo, Palo Alto, CA (US); Kendra Dee Nyberg, Corvallis, OR (US); Milo Overbay, Corvallis, OR (US)

(73) Assignee: Hewlett-Packard Development Company, L.P., Spring, TX (US)

(21) Appl. No.: 17/047,336

(22) PCT Filed: Aug. 21, 2018
(86) PCT No.: PCT/US2018/047343

§ 371 Date: Oct. 13, 2020

(51) Int. Cl. C12M 3/06; G01N 15/02; G01N 15/06; G01N 15/14

(52) U.S. Cl. CPC C12M 23/16; G01N 15/0227; G01N 2015/0061; G01N 15/1475; G01N 15/0612

(57) ABSTRACT

An example of an apparatus includes an inlet to receive a plurality of cells suspended in a solution. The apparatus also includes a microfluidic channel to transport the plurality of cells suspended in the solution. In addition, the apparatus includes a trap disposed along the microfluidic channel, wherein the trap is to isolate the plurality of cells suspended in the solution. Also, the apparatus includes a buffer supply to dispense a buffer to wash the plurality of cells and to remove the solution from the microfluidic channel. The apparatus further includes a sensor to measure a characteristic of the plurality of cells after isolated from the solution.

### BACKGROUND

Isolating cells for measurements may be used in various industries, such as biology and medicine. For example, cells may be counted or turbidity may be measured to determine the density of cells in a given volume. This may provide an ability to make evaluations in several different applications. For example, measuring cells may have applications in antimicrobial susceptibility testing, such as for determining a minimum inhibitory concentration.

### BRIEF DESCRIPTION OF THE DRAWINGS

Reference will now be made, by way of example only, to the accompanying drawings in which:

FIG. 1 is a schematic diagram of an example apparatus to isolate cells from a mixture and measure characteristics;

FIG. 2 is a schematic diagram of another example apparatus to isolate cells from a mixture and measure characteristics;

FIG. 3 is a schematic diagram of the apparatus shown in FIG. 2 receiving a mixture;

FIG. 4 is a schematic diagram of the apparatus shown in FIG. 2 isolating cells and incubating the cells;

FIG. 5 is a schematic diagram of the apparatus shown in FIG. 2 measuring a characteristic;

FIG. 6 is a schematic diagram of another example of a trap to isolate cells;

FIG. 7 is a schematic diagram of another example apparatus to isolate cells from a mixture; and

FIG. 8 is flowchart of an example method of isolating cells from a mixture and measure characteristics.

### DETAILED DESCRIPTION

Measurements of cells may have many applications and may involve many techniques. For example, an application of measuring cells may be to determine bacteria cell health during antimicrobial susceptibility testing, such as to determine a minimum inhibitory concentration of an antibiotic during a testing phase for an antibiotic. One method to determine the minimum inhibitory concentration involves dispensing antibiotics of varying concentration into separate wells containing bacteria. Each well may then be monitored, such as by observing the turbidity in the well. It is to be appreciated that by using this method, the minimum inhibitory concentration may be determined after sufficient time elapses that enough cells grow in a well for a reliable positive turbidity measurement, which may be about 24 to 48 hours. In other examples, rapid minimum inhibitory concentration determination may be made by directly measuring biomarkers indicating cell health using a spectroscopic technique such as surface-enhanced Raman spectroscopy or surface-enhanced Infrared absorption spectroscopy.

Furthermore, by using a spectroscopic technique, such as surface-enhanced Raman spectroscopy or surface-enhanced Infrared absorption spectroscopy, the measurements may be made in a microfluidic or nanofluidic platform instead of conventional wells. Microfluidic and nanofluidic platforms may be used to manipulate and sample small amounts of colloids, inert particles, and biological microparticles, such as red blood cells, white blood cells, platelets, cancer cells, bacteria, yeast, microorganisms, proteins, DNA, etc. Accordingly, less time may be involved in growing a sufficient sample size. In addition, the apparatus used to make the measurements may be smaller.

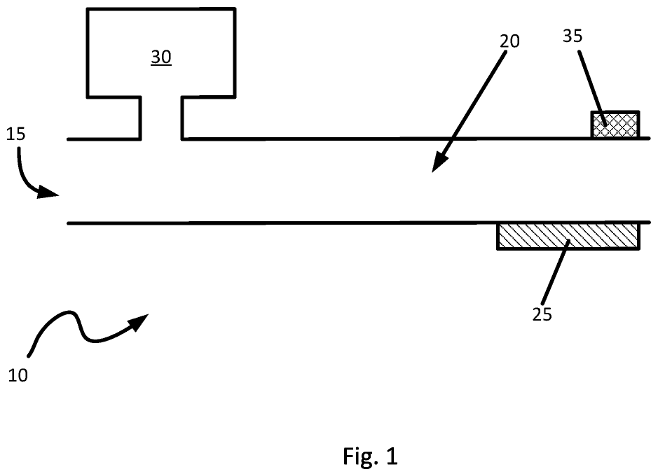

Referring to FIG. 1, an apparatus to isolate cells from a solution and measure characteristics of the isolated cells is shown at 10. The apparatus 10 is to receive a plurality of cells in a solution for separation and measurement. In the present example, the apparatus 10 includes an inlet 15, a microfluidic channel 20, a trap 25, a buffer supply 30, and a sensor 35.

The inlet 15 is to receive a mixture which includes a plurality of cells suspended in a solution. The plurality of cells is not limited and may include several different types of cells. In the present example, the plurality of cells includes a plurality of bacteria. In particular, the bacteria in the present example may be substantially all of the same type, such as in a culture of bacteria. In other examples, the plurality of cells may be other types of cells, such as cells from an animal or human. For example, the plurality of cells may include red blood cells, white blood cells, platelets, cancer cells, and/or yeast. In further examples, the plurality of cells may also be substituted with other biological materials that may be parts of cells, such as proteins, DNA, RNA, exosomes, and other biological microparticles, or a small collection of cells, such as small microorganisms.

The source of the plurality of cells is not particularly limited. For example, the plurality of cells may be suspended in a solution stored in an external well or reservoir (not shown). The inlet 15 may then draw the fluid into the apparatus 10 with capillary action or with a pump (not shown) or other means. In other examples, the plurality of cells may be received from an external dispensing mechanism or directly from a sample collected from a bacteria culture or from a patient. The sample size of the plurality of cells flow in the solution is not particularly limited. In the present example, the sample size is about 10 to 100 cells. In other examples, the sample size may be increased to about 1000 cells or decreased to a single cell. It is to be appreciated that other examples having different configurations may allow for large or smaller sample sizes beyond the range.

The solution in which the plurality of cells is mixed is not particularly limited. In the present example, the solution may include a dose of an antibiotic, drug, or another medical component. Accordingly, the solution may be used to administer the medical component, such as an antibiotic, to the cells prior to arrival at the inlet 15. The manner by which the plurality of cells interacts with the medical component prior to arrival at the inlet 15 is not limited and may involve mixing the cells and the solution is a separate container for an amount of time. In other examples, the solution may contain chemotherapy drugs or unique nutrient mixtures. In further examples, the mixture received at the inlet 15 may be a direct tissue sample, such as blood.

In the present example, the microfluidic channel 20 is to transport the plurality of cells suspended in the solution. In the present example, the microfluidic channel 20 is about 10pm to 100pm wide by about 100pm tall. In other examples, it is to be appreciated that the microfluidic channel 20 may be replaces with a nanofluidic channel to draw and smaller sample size of cells and solution.

The trap 25 is disposed along the microfluidic channel 20. In the present example, the trap 25 is to isolate the plurality of cells suspended in the solution. In particular, the trap 25 is to effectively separate the plurality of cells from the solution in which the plurality of cells was suspended. It is to be appreciate that as the mixture of the plurality of cells and solution move through the microfluidic channel 20, the trap may separate the plurality of cells by attracting or otherwise inhibiting cells from moving through the microfluidic channel while allowing the solution to continue flowing through.

In an example, the mixture of the plurality of cells and solution may also include a magnetic material, such as magnetic beads, dispersed homogenously throughout the mixture. The magnetic beads are not particularly limited and may include any ferromagnetic or superparamagnetic material, such as iron, iron oxide, chromium oxide, nickel, and cobalt. Furthermore, the size of the magnetic beads is not limited. For example, the magnetic beads may be substantially uniform in size or may include a distribution of sizes. In addition, the dimensions of the magnetic beads may be selected based on the application, such as the size of an average cell in the plurality cells. In some examples, the magnetic beads may also have varying shapes or may include a rough surface to promote interaction with the plurality of cells.

In the present example, the magnetic beads may also be coated with a protective layer to reduce a potential reaction between the magnetic beads and the plurality of cells or the solution. Some examples of a protective layer may be a silica, plastic, or parylene material. In this example, the trap 25 may include a magnet that may be controlled to attract the magnetic beads to a side of the microfluidic channel 20. Since the magnetic beads are dispersed among the plurality of cells, the magnetic beads may serve to hold cells from the plurality of cells against the wall of the microfluidic channel 20. Accordingly, the magnetic beads may include surface features, such as roughness or adhesiveness, to promote the interaction or binding between the magnetic bead and the cells. In addition, the magnet may be designed to interact with the magnetic beads to provide sufficient force to hold the magnetic beads and the plurality of cells in place proximate to the trap 25. It is to be appreciated that the solution is not affected by the movements magnetic beads and may continue to flow around the cells and the magnetic beads once the trap 25 engages the magnetic beads.

The buffer supply 30 is to dispense a buffer into the microfluidic channel 20. In the present example, the buffer supply 30 is to be connected to the microfluidic channel 20 and controlled to dispense the buffer during a washing phase. The buffer dispensed by the buffer supply is not particularly limited and may include water, phosphate buffered saline, cholamine chloride or tris(hydroxymethyl)aminomethane. The buffer may be to remove the solution from the original mixture to remove additional molecules or biomarkers that may affect the sensor 35. For example, the original solution may include an antibiotic that may provide a separate response to the sensor 35 that may mask the signal of a specific biomarker to be monitored.

In another example, the buffer may be selected to induce a stress response from the plurality of cells to increase the prominence of the biomarker. In examples, where the health of the cells is to be measured, the buffer may be selected to induce different responses from the cells dependent on the health of the cell. For example, the buffer may be a solution that induces a stress response from healthy cells such as deionized water with no nutrients and/or low molality to provide an increase in a biomarker, such as adenine, xanthine and hypoxanthine. In this example, dead or diseased cells trapped in the microfluidic channel 20 may provide no significant response. Therefore, signals provided by a biomarker may be subsequently measured to determine the health of the plurality of cells. For example, the intensity of a signal associated with a biomarker may provide an indication of the amount of healthy cells in a sample.

The sensor 35 is to measure a characteristic of the plurality of the cells. In the present example, the sensor is to measure the characteristic after the cells are isolated from the original solution, such as after the buffer has washed the cells isolated and held by the trap 25. It is to be appreciated that the sensor 35 is not particularly limited and may be selected based on the characteristics of the cells that are to be measured. In the present example, the characteristic to be measured may be associated with a cell count or other indication of the heath of a sample of the cells. This characteristic may be used to determine the effects of a medical component, such as an antibiotic, in the original solution prior to arrival at the inlet 15. The health of the plurality of cells may then be used to determine an effective dose of the medical component or a minimum inhibitory concentration of the antibiotic.

The sensor 35 is not limited and may be any type of sensor capable of measuring a desired characteristic of the plurality of cells. In the present example, the sensor 35 may be a spectrometer for detecting signals from a light source to detect spectroscopic signals that may be reflected or transmitted through the plurality of cells. For example, the sensor 35 may be a Raman spectrometer to carry out surface-enhanced Raman spectroscopy after a monochromatic light source, such as a laser, emits light on the plurality of cells. This technique may be used to detect the presence of biomarkers produced by healthy cells to provide an indication as to the health of the cells. As discussed above, a buffer may also be selected that may induce additional biomarker production by the healthy cells to increase the intensity of a response during the detection of the characteristic. As another example, the sensor 35 may be an infrared spectrometer to carry out surface-enhanced infrared absorption spectroscopy after exposing the cells to infrared radiation. This technique may also be used to detect the presence of biomarkers produced by healthy cells to provide an indication as to the health of the cells. In yet another example, the sensor 35 may be a combination of both a Raman spectrometer and an infrared detector, such that characteristics of the cells may be detected using multiple methods.

Although FIG. 1 shows the sensor 35 located proximate to the trap 25 on the microfluidic channel 20, the location of the sensor 35 is not particularly limited. In the present example, the sensor 35 is proximate to the trap 25 such that the sensor 35 may measure a characteristic of the cells while the cells are held by the trap 25 after the cells are isolated from the original solution by the buffer. It is to be appreciated that in this example of using magnetic beads where the plurality of cells is held by the trap 25, the magnetic beads will be mixed with the cells during a measurement process. The magnetic beads may introduce artifacts into the signals detected by the sensor 35. In other examples, the sensor 35 may be located away from the trap 25 such that the magnetic beads may be separated and removed from the cells prior to measurement of characteristics of the cells. The manner by which the magnetic beads is released is not limited and may involve releasing the magnetic beads from the trap 25 by turning off the magnet. Subsequently, the magnetic beads may be separated from the cells using mechanical methods such as filters or other separation techniques and transported to the sensor 35.

In another example, the sensor 35 may be used to measure the characteristics of the buffer instead of the cells. In this example, the cells may remain held by the trap 25 and the buffer used to wash the cells may be collected and analyzed using the sensor 35. Since the magnetic beads and the cells remain held by the trap 25, biomarkers and other molecules that may provide an indication of the health of the cells may separate and be carried by the buffer. Accordingly, this manner of analysis may provide a better sample free from artifacts that may be introduced by other portions of the cell, the magnetic beads, and/or the trap 25.

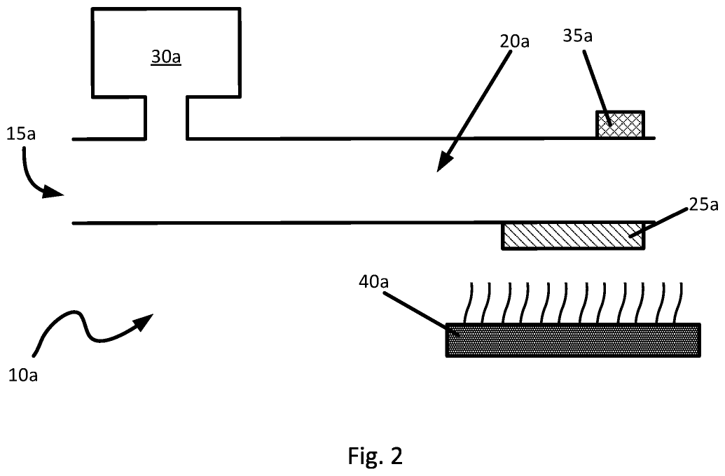

Referring to FIG. 2, another example of an apparatus to isolate cells from a solution and measure characteristics of the isolated cells is shown at 10a. Like components of the apparatus 10a bear like reference to their counterparts in the apparatus 10, except followed by the suffix “a”. The apparatus 10a includes an inlet 15a, a microfluidic channel 20a, a trap 25a, a buffer supply 30a, a sensor 35a, and a heating element 40a.

In the present example, the apparatus 10a includes a heating element 40a to provide heat to the plurality of cells in the microfluidic channel 20a. In this example, the heating element 40a is to incubate the cells to promote interactions between the cells and the buffer, such as to increase the rate at which material, such as biomarkers, is transferred to the buffer. In other examples, the heating element 40a may also be used to increase the rate at which a stress response is induced by the buffer. In the present example, the heating element 40a is proximate to the trap 25a and is to incubate the cells that are held by the trap 25a. In other examples, the heating element 40a may heat the entire apparatus 10a such that the cells may be incubated prior to isolation and washing to provide additional interactions between the cells and the original solution.

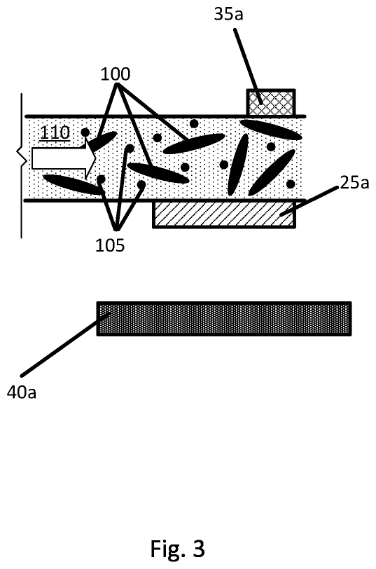

Referring to FIG. 3, the apparatus 10a is shown in operation. In the present example, a mixture of bacteria 100 and magnetic beads 105 in a solution 110 is received into the microfluidic channel 20a. In this example, the bacteria 100 and the magnetic beads 105 are in a homogenous mixture. In other examples, the magnetic beads 105 may be bound to the bacteria 100. In further examples, the magnetic beads 105 may be introduced into the microfluidic channel 20a after the introduction of the bacteria 100.

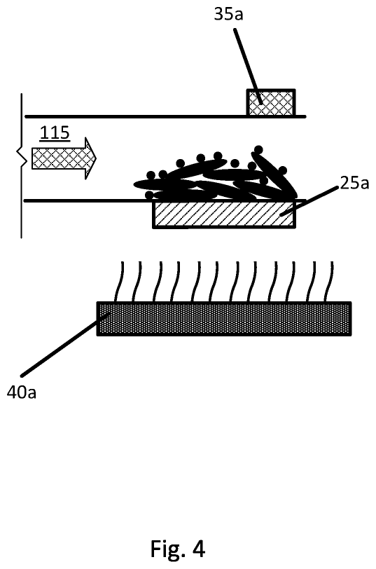

Next, referring to FIG. 4, the trap 25a is turned on to create a magnetic field. The magnetic field is to attract the magnetic beads 105 in the mixture. As the magnetic beads 105 are attracted to the trap 25a, the magnetic beads 105 may push the bacteria 100 to the trap 25a and hold the bacteria 100 against the wall of the microfluidic channel 20a. A buffer 115 may then be passed over the bacteria 100 to isolate the bacteria 100 from any residual solution 110 remaining on the surface of the bacteria 100. In addition, the heating element 40a may be used to incubate the cells held by the trap 25a.

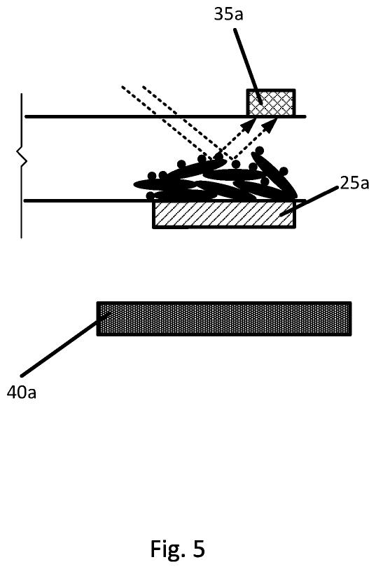

FIG. 5 shows the sensor 35a in operation to measure a characteristic of the cells. In the present example, a light source (not shown) directs light to the plurality of cells at the trap 25. The sensor 35a may receive light that is reflected off the cells or off a substrate material. In this example, the sensor 35a is a Raman spectrometer to carry out surface-enhanced Raman spectroscopy after a monochromatic light source, such as a laser, emits light on the plurality of cells. This technique may be used to detect the presence of biomarkers produced by healthy cells to provide an indication as to the health of the cells. As discussed above, a buffer may also be selected that may induce additional biomarker production by the healthy cells to increase the intensity of a response during the detection of the characteristic.

In other examples, the sensor 35a may be use additional and/or alternative sensing techniques to measure the characteristic. For example, additional measurements may be based on real time microscopic image inspection for changes in size, shape, number, changes in impedence to indicate cell health, flow cytometry fluorescent tags, or microcantilever weighing methods.

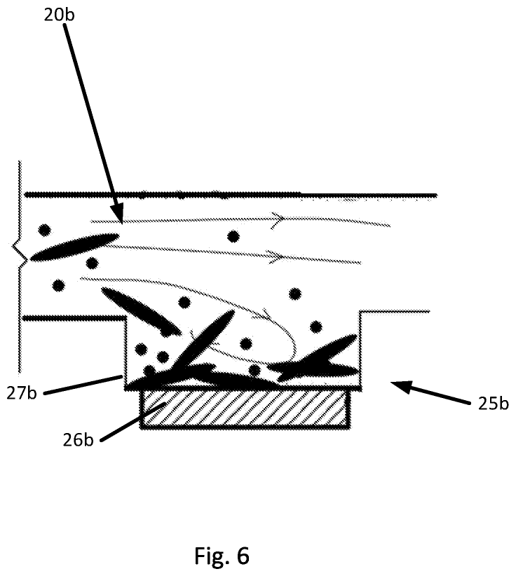

Referring to FIG. 6, another example of a trap 25b using inertial microfluidics channel may be used to enhance the trapping efficiency of the magnet 26b. For example, a step feature 27b of about 20 μm to about 70 μm in the microfluidic channel 20 may create a vortex in the microfluidic channel 20b. Accordingly, particles with higher inertia tend to circulate into the eddy at the step feature 27b as fluid flows past the step feature 27b. It is to be appreciated that the physical sorting based on size enables bacteria 100 to spend more time in the proximity of the magnet. In other examples, the magnet 26b may be omitted such that the trap 25b includes the step feature 27b alone.

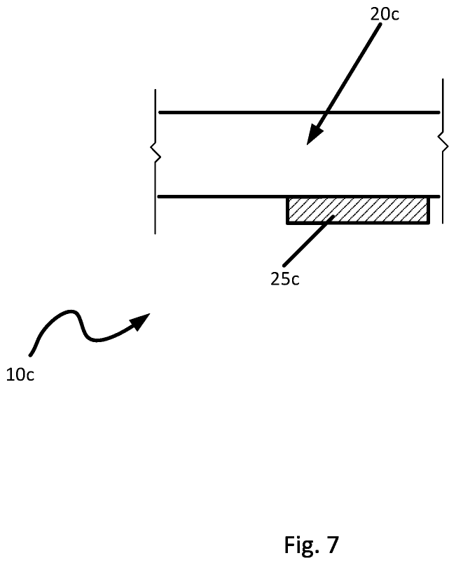

Referring to FIG. 7, another example of an apparatus to isolate cells from a solution and measure characteristics of the isolated cells is shown at 10c. Like components of the apparatus 10c bear like reference to their counterparts in the apparatus 10, except followed by the suffix “c”. The apparatus 10c includes a microfluidic channel 20c, and a magnet 25c,

In the present example, the microfluidic channel 20c is to receive a mixture of bacteria and magnetic beads suspended in a solution. In the present example, the solution includes an antibiotic dose. Accordingly, the solution may be used to administer the antibiotic dose to the bacteria in the mixture to test the effectiveness of the antibiotic. The manner by which the bacteria interacts with the antibiotic prior to being received in the microfluidic channel 20c is not limited and may involve adding the solution to a bacteria culture. The mixture may also be incubated prior to entering the microfluidic channel 20c or while the mixture is in the microfluidic channel 20c.

The magnet 25c is disposed along the microfluidic channel 20c. In the present example, the magnet 25c is to interact with the magnetic beads suspended in the solution. The magnet 25c is not particularly limited and may be a permanent magnet, such as a ferromagnetic material, or an electromagnet. In particular, the magnet 25c is to effectively separate the bacteria from the solution in which the bacteria are suspended. It is to be appreciate that as the mixture of the bacteria and solution move through the microfluidic channel 20c, the magnet 25c may attract the magnetic beads to hold them close to the wall of the microfluidic channel 20c. In this example, the magnetic beads will be used to trap bacteria against the wall due to the magnetic force exerted by the magnet 25c.

Once the bacteria are isolated against the wall of the microfluidic channel 20 by the magnet 25c interacting with the magnetic beads, a buffer may be used to wash the bacteria to remove any residual solution in the microfluidic channel 20c as well as on the surface of the bacteria. This may be useful if the dose of antibiotics involves a controlled time period such that interactions between the antibiotic and the bacteria is to be stopped. A characteristic of the bacteria may then be measured after the bacteria is washed. In the present example, the characteristic is measured using a spectrometer after exposing the bacteria to a light source. It is to be appreciated that this measure may be made while the bacteria is held against the wall of the microfluidic channel 20c by the magnetic beads at the magnet 25c. Accordingly, the entire platform holding the microfluidic channel 20c may be placed within the spectrometer. In other examples, the magnet 25c may release the bacteria after washing for transport to a spectrometer. The spectrometer may be used to measure specific biomarkers of the bacteria to evaluate the health of the bacteria and determine whether the antibiotic dose administered meets the minimum inhibitory concentration threshold.

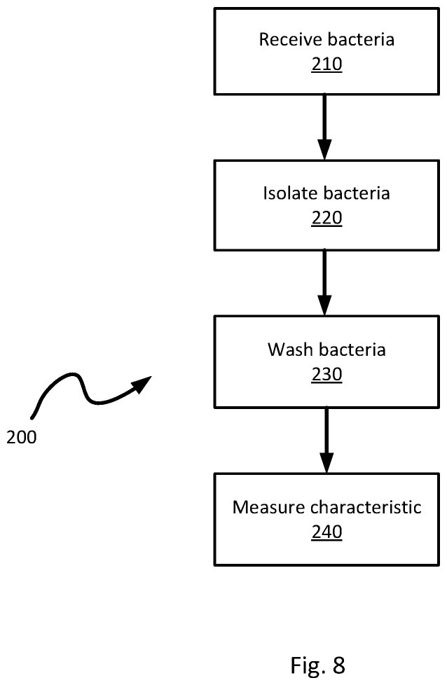

Referring to FIG. 8, a flowchart of a method of isolating cells from a solution and measuring characteristics of the isolated cells is shown at 200. In order to assist in the explanation of method 200, it will be assumed that method 200 may be performed with any of the apparatus 10, 10a, or 10c described above. Indeed, the method 200 may be one way in which apparatus 10, 10a, or 10c may be configured to isolate cells from a solution and measuring a characteristic of the cells. Furthermore, the following discussion of method 200 may lead to a further understanding of the apparatus 10, 10a, or 10c and their various components. For purposes of the following discussion, it is to be assumed that the method 200 is carried out on the apparatus 10. Furthermore, it is to be emphasized, that method 200 may not be performed in the exact sequence as shown, and various blocks may be performed in parallel rather than in sequence, or in a different sequence altogether.

Beginning at block 210, a mixture of bacteria suspended in a solution is received at the apparatus via a microfluidic channel 20. The solution in which the bacteria is mixed is not particularly limited. In the present example, the solution is to provide a treatment to the bacteria. For example, the treatment may include administering an antibiotic to kill the bacteria cells. It is to be appreciated that the solution may have administered a treatment to the bacteria prior to arrival at the microfluidic channel 20. In other examples, the treatment may be administered while in the microfluidic channel 20.

Block 220 involves isolating the bacteria in the microfluidic channel 20 using a trapping mechanism such as the trap 25 shown in FIG. 1. In an example, the mixture of bacteria suspended in a solution may also include a magnetic material dispersed throughout the mixture. The magnetic material is not particularly limited and may include a ferromagnetic or superparamgnetic material. Furthermore, the size of the magnetic material is not limited. In some examples, the magnetic material may be absorbed by the bacteria. In some examples, the magnetic material may have varying shapes or may include a rough surface or features to promote interaction with the plurality of cells.

Block 230 washes the bacteria with a buffer to remove the solution. The manner by which the bacteria is washed is not limited and may involve flowing buffer over the bacteria held by the trap 25. In the present example, the buffer provided may include a substance that may be used to induce a response from healthy bacteria cells. For example, the response may be to produce additional biomarkers for detection.

Next, block 240 involves measuring a characteristic of the bacteria. In the present example, the characteristic to be measured may be an indicator of bacteria health. In this example, the characteristic may be used to determine the effectiveness of a treatment. For example, if the bacteria are treated with an antibiotic, the characteristic may be a signal from a spectroscopy technique associated with a biomarker generated by the bacteria. Accordingly, if the intensity of the signal is weak, it may provide an indication that the number of bacteria is low and/or the bacteria is no longer alive. Conversely, if the intensity of the signal is strong, it may provide an indication that the number of bacteria is high and/or the bacteria are still alive. The measurements are not particularly limited. For example, the measurements may involve performing surface-enhanced Raman spectroscopy on the bacteria to look for biomarkers or performing surface-enhanced infrared spectroscopy on the bacteria to look for biomarkers.

It should be recognized that features and aspects of the various examples provided above may be combined into further examples that also fall within the scope of the present disclosure.

What is claimed is:

1. An apparatus comprising:
an inlet to receive a plurality of cells suspended in a solution;
a microfluidic channel to transport the plurality of cells suspended in the solution;
a trap disposed along the microfluidic channel, wherein the trap is to isolate the plurality of cells suspended in the solution;
a buffer supply to dispense a buffer to wash the plurality of cells and to remove the solution from the microfluidic channel; and
a sensor to measure a characteristic of the plurality of cells after isolated from the solution.

2. The apparatus of claim 1, wherein the solution includes an antibiotic.

3. The apparatus of claim 2, wherein the characteristic is to indicate a cell health to determine a minimum inhibitory concentration of the antibiotic.

4. The apparatus of claim 1, wherein the sensor is to measure the characteristic of the plurality of cells at the trap.

5. The apparatus of claim 1, wherein the trap comprises a magnet to interact with magnetic beads, wherein the magnetic beads are dispersed among the plurality of cells.

6. The apparatus of claim 5, wherein the sensor is to measure the characteristic of the plurality of cells away from the trap after removal of the magnetic beads.

7. The apparatus of claim 1, wherein the sensor is to measure the characteristic of the plurality of cells via the buffer used to wash the plurality of cells.

8. The apparatus of claim 7, further comprising a heating element to incubate the plurality of cells in the buffer to promote transfer of material from the plurality of cells to the buffer.

9. A method comprising:
receiving bacteria suspended in a solution via a microfluidic channel, wherein the solution is to provide a treatment;
isolating the bacteria in the microfluidic channel with a trapping mechanism;
washing the bacteria with a buffer to remove the solution; and
measuring a characteristic of the bacteria.

10. The method of claim 9, wherein providing the treatment comprises administering an antibiotic in the solution.

11. The method of claim 10, wherein measuring the characteristic comprises measuring an indication of bacteria health to determine minimum inhibitory concentration of the antibiotic.

12. The method of claim 11, wherein measuring the characteristic comprises performing surface-enhanced Raman spectroscopy on the bacteria.

13. The method of claim 11, wherein measuring the characteristic comprises performing surface-enhanced infrared absorption spectroscopy on the bacteria.

14. An apparatus comprising:
a microfluidic channel to receive a mixture of bacteria and magnetic beads suspended a solution, wherein the solution includes an antibiotic dose; and
a magnet disposed along the microfluidic channel, wherein the magnet is to interact with the magnetic beads, wherein the magnet attracts the magnetic beads to isolate the bacteria against a wall of the microfluidic channel,
wherein the microfluidic channel receives a buffer to remove the solution from the microfluidic channel when the bacteria is isolated against the wall by the magnet to allow a spectrometer to measure a characteristic of the bacteria after isolation from the solution.

15. The apparatus of claim 14, wherein the spectrometer determines a heath of the bacteria to determine whether the antibiotic dose provides a minimum inhibitory concentration.
